(19) United States
(12) Patent Application Publication
(10) Pub. No.: US 2026/0308025 A1
(43) Pub. Date: Oct. 8, 2026

(54) Contaminant Precipitation Compositions and Methods of Use

(71) Applicant: Locus Solutions IPCo, LLC, Solon, OH (US)

(72) Inventors: Gabriela KNESEL, Solon, OH (US); Ronney SILVA, Solon, OH (US); Cathrine MONYAKE, Solon, OH (US); Juan CERVANTES, Solon, OH (US)

(21) Appl. No.: 18/880,479

(22) PCT Filed: Sep. 18, 2024
(86) PCT No.: PCT/US2024/047245

§ 371 Date: Dec. 31, 2024

Related U.S. Application Data
(60) Provisional application No. 63/583,634, filed on Sep. 19, 2023.

(51) Int. Cl. C02F 1/54; C02F 1/52; C02F 103/10

(52) U.S. Cl. CPC C02F 1/547; C02F 1/5245; C02F 2103/10

(57) ABSTRACT

The subject invention provides safe, environmentally-friendly compositions and efficient methods of precipitating contaminants, target elements, targe minerals, and other target compounds out of a liquid. More specifically, the subject invention provides compositions derived from microorganisms for precipitation, which can be used for increasing the rate of precipitation, the quality of the precipitant, and/or the amount of precipitant.

### CROSS-REFERENCE TO RELATED APPLICATION

This application claims priority to U.S. Provisional Patent Application No. 63/583,634, filed Sep. 19, 2023, which is incorporated herein by reference in its entirety.

### BACKGROUND OF THE INVENTION

Precipitation reactions occur when cations and anions in an aqueous solution combine to form an insoluble ionic precipitate. Reactions can depend on temperature, buffering solutions, and the concentration of solutes in the solvent. The precipitates formed in these reactions are crystalline solids, and the remaining liquid is the supernatant liquid or the supernate. The precipitate and the supernate can be separated by various methods, such as, for example, filtration, centrifugation, or decanting.

Most precipitation reactions are single replacement or double replacement reactions. A double replacement reaction describes the dissociation of two ionic reactants and the bonding of the dissociated ionic reactants with distinct anions or cations.

Precipitation reactions can be used to determine the presence of an element in a solution. For example, a precipitation reaction can be used to determine the presence of lead in water sources. Precipitation reactions can also be used to extract elements, including, for example, magnesium from sea water.

Oil and gas production wells, mining sites, and quarrying sites generate large volumes of water mixed with hydrocarbons (dispersed and dissolved), salts (e.g., sodium, calcium, magnesium, lithium, sulfates, and carbonates), minerals, elements, compounds, and metals. Additionally, industrial wastewater can also contain valuable elements, compounds, metals, and minerals, including lithium and rare earth elements. The metals, minerals, elements, compounds, and other material can be used to create valuable products, including, for example, lithium-based batteries and electronics. Chemical precipitation is commonly used to treat mining-influenced water (MIW), including, for example, acid mine drainage, neutral drainage, and pit lake water. These reactions can be performed in a clarifier or by filtration with ceramic membranes or other membranes.

Chemical precipitation can be used to extract contaminants, elements, minerals, and resources from wastewater; however, existing methods of precipitation are expensive and unreliable. Therefore, novel, improved methods are needed for the separation of contaminants, target elements, target minerals, or target compounds from liquids by precipitation.

### BRIEF SUMMARY OF THE INVENTION

The subject invention relates generally to precipitation compositions and methods of using these compositions. More specifically, the subject invention provides environmentally-friendly methods of precipitation, such as, for example, precipitating solids from mining waste, including acid mine drainage, neutral drainage, and pit lake water. In certain embodiments, existing methods can incorporate the subject compositions and methods. Alternatively, in certain embodiments, the subject methods can replace existing methods of contaminant precipitation.

Advantageously, the composition and methods of the subject invention can increase the efficiency of precipitation and can decrease chemical usage, including chemical surfactant usage that can be required for the precipitation of contaminants, target elements, target minerals, or other target compounds.

In certain embodiments, the subject invention provides compositions comprising components that are derived from microorganisms. In certain embodiments, the composition comprises a microbial biosurfactant. In certain embodiments, the composition comprises one or more biosurfactants, and, optionally, other compounds, such as, for example, water; alkali; sulfide; coagulant; chemical surfactant, including, for example, an ionic surfactant or a nonionic surfactant; flocculant; clarifying agent; filtration aid; or any combination thereof.

In certain embodiments, the subject compositions can be applied to the mining waste or a slurry of ore directly. In some embodiments, mixing the subject composition with the mining waste or slurry of ore can create a precipitate, which can be separated from the mining waste or a slurry of ore.

In certain embodiments, the subject composition can be applied directly to, for example, a clarifier or other vessel in which a liquid is treated.

In certain embodiments, the subject compositions and methods can be used to precipitate contaminants, target elements, target minerals, or other target compounds in water from, for example, mining sites, quarrying sites, oil and gas sites, mineral refineries, wastewater sites, agricultural sites, and food and beverage production sites. The subject compositions can be used treat multiple contaminants of concern, such as, for example, heavy metals, salts, oils, and/or greases.

In certain embodiments, the biosurfactant of the composition is utilized in crude form. The crude form can comprise, in addition to the biosurfactant, fermentation broth in which a biosurfactant-producing microorganism was cultivated, residual microbial cell matter or live or inactive microbial cells, residual nutrients, and/or other microbial growth by-products.

In some embodiments, the biosurfactant is utilized after being extracted from a fermentation broth and, optionally, purified.

The biosurfactant according to the subject invention can be a glycolipid (e.g., sophorolipids, rhamnolipids, cellobiose lipids, mannosylerythritol lipids and trehalose lipids), lipopeptide (e.g., surfactin, iturin, fengycin, arthrofactin, and lichenysin), flavolipids, phospholipid (e.g., cardiolipins), fatty acid ester compound, fatty acid ether compound, and/or high molecular weight polymers such as lipoproteins, lipopolysaccharide-protein complexes, and polysaccharide-protein-fatty acid complexes.

In certain specific embodiments, the biosurfactant is a sophorolipid (SLP), including linear SLP, lactonic SLP, acetylated SLP, de-acetylated SLP, salt-form SLP, esterified SLP derivatives, amino acid-SLP conjugates, and other SLP derivatives or isomers that exist in nature and/or are produced synthetically. In preferred embodiments, the SLP is a linear SLP or a derivatized linear SLP.

In certain embodiments, the subject invention provides a method of precipitation, wherein the method comprises the following steps:
a) contacting the precipitation composition comprising a biosurfactant with a liquid to yield a precipitate and the liquid; and
b) separating the precipitate from the liquid.

In some embodiments, the biosurfactant is a charged biosurfactant. In certain embodiments, the liquid contains dissolved ionic molecules. In certain embodiments, the reactants in the precipitation reaction form a neutral charged precipitate.

In certain embodiments, the mixture can be agitated during step a). In certain embodiments, the removal of the precipitate from the liquid can be performed using a pressurized flow of a liquid, centrifugation, or gravitational principals, filtration, or any combination thereof.

In some embodiments the precipitate particles can be less than about 10 cm, less than about 1 cm, less than about 1 mm, less than about 500 μm, less than about 100 μm, less than about 10 μm, less than about 1 μm, less than about 100 nm, less than about 10 nm, or less than about 1 nm in diameter.

In some embodiments, the method comprises contacting a precipitation composition comprising a biosurfactant to a liquid containing dissolved molecules, and, optionally, other components, such as, for example, water; alkali; sulfide; coagulant; chemical surfactants, including, for example, ionic surfactants or nonionic surfactants; flocculants; clarifying agents; filtration aids; or any combination thereof. In certain embodiments, the precipitation composition is contacted to the liquid for a period of time and/or until a distinct volume of the precipitation composition has been contacted to the liquid. This step can be repeated as many times as necessary to achieve a rate of precipitation or until a desired amount of solid particles are removed from the liquid.

Advantageously, in certain embodiments, the precipitation composition according to the subject invention can be effective at precipitating toxic solids. Furthermore, the methods of the subject invention do not require complicated equipment or high energy consumption, and production of the composition can be performed on site, for example, at a mine or at a wastewater treatment facility.

### DETAILED DESCRIPTION

The subject invention relates generally to the precipitation of contaminants, target elements, target minerals, or other target compounds out of liquids. More specifically, the subject invention provides environmentally-friendly compositions and methods of precipitation, such as, for example, precipitating metals from liquids that are produced at mining sites, found in wastewater, or liquids derived from or used in industrial activities. Accordingly, the subject invention is useful for improving the efficiency and efficacy of methods of precipitation. Advantageously, the compositions and methods of the subject invention increase the efficiency of the precipitation of solids using safe, environmentally-friendly compositions.

### Selected Definitions

As used herein, “applying” a composition or product refers to contacting it with a target or site such that the composition or product can have an effect on that target or site. The effect can be due to, for example, microbial growth and/or the action of a biosurfactant or other microbial growth by-product.

As used herein, a “biofilm” is a complex aggregate of microorganisms, such as bacteria, yeast, or fungi, wherein the cells adhere to each other and/or to a surface using an extracellular matrix. The cells in biofilms are physiologically distinct from planktonic cells of the same organism, which are single cells that can float or swim in liquid medium.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An isolated microbial strain means that the strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 98%, by weight the compound of interest. For example, a purified compound is one that is at least 80%, 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism or a substance necessary for taking part in a particular metabolic process. A metabolite can be an organic compound that is a starting material, an intermediate in, or an end product of metabolism. Examples of metabolites include, but are not limited to, enzymes, acids, solvents, alcohols, proteins, vitamins, minerals, microelements, amino acids, biopolymers and biosurfactants.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

As used herein, “surfactant” means a compound that lowers the surface tension (or interfacial tension) between two liquids or between a liquid and a solid. Surfactants act as, e.g., detergents, wetting agents, emulsifiers, foaming agents, and/or dispersants. A “biosurfactant” is a surface-active substance produced by a living cell and/or using naturally-derived substrates.

Biosurfactants are a structurally diverse group of surface-active substances consisting of two parts: a polar (hydrophilic) moiety and non-polar (hydrophobic) group. Due to their amphiphilic structure, biosurfactants can, for example, increase the surface area of hydrophobic water-insoluble substances, increase the water bioavailability of such substances, and change the properties of bacterial cell surfaces. Biosurfactants can also reduce the interfacial tension between water and oil and, therefore, lower the hydrostatic pressure required to move entrapped liquid to overcome the capillary effect. Biosurfactants accumulate at interfaces, thus reducing interfacial tension and leading to the formation of aggregated micellar structures in solution. The formation of micelles provides a physical mechanism to mobilize, for example, oil in a moving aqueous phase.

The ability of biosurfactants to reduce the surface tension also permits their use as antibacterial, antifungal, and hemolytic agents to, for example, control pests and/or microbial growth.

Typically, the hydrophilic group of a biosurfactant is a sugar (e.g., a mono-, di-, or polysaccharide) or a peptide, while the hydrophobic group is typically a fatty acid. Thus, there are countless potential variations of biosurfactant molecules based on, for example, type of sugar, number of sugars, size of peptides, which amino acids are present in the peptides, fatty acid length, saturation of fatty acids, additional acetylation, additional functional groups, esterification, polarity and charge of the molecule.

These variations lead to a group of molecules comprising a wide variety of classes, including, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes. Each type of biosurfactant within each class can further comprise subtypes having further modified structures.

Like chemical surfactants, each biosurfactant molecule has its own HLB value depending on its structure; however, unlike production of chemical surfactants, which results in a single molecule with a single HLB value or range, one cycle of biosurfactant production typically results in a mixture of biosurfactant molecules (e.g., subtypes and isomers thereof).

The phrases “biosurfactant” and “biosurfactant molecule” include all forms, analogs, orthologs, isomers, and natural and/or anthropogenic modifications of any biosurfactant class (e.g., glycolipid) and/or subtype thereof (e.g., sophorolipid).

As used herein, the term “sophorolipid,” “sophorolipid molecule,” “SLP” or “SLP molecule” includes all forms, and isomers thereof, of SLP molecules, including, for example, acidic (linear) SLP (ASL) and lactonic SLP (LSL). Further included are mono-acetylated SLP, di-acetylated SLP, esterified SLP, SLP with varying hydrophobic chain lengths, cationic and/or anionic SLP with fatty acid-amino acid complexes attached, esterified SLP, SLP-metal complexes, SLP-salt derivatives (e.g., a sodium salt of a linear SLP), and other, including those that are and/or are not described within in this disclosure.

In certain embodiments, the glycolipid biosurfactant is a sophorolipid (SLP). Sophorolipids are glycolipid biosurfactants produced by, for example, various yeasts of the Starmerella clade when cultivated in the presence of a hydrocarbon-based source of one or more fatty acids. SLP typically consist of a disaccharide sophorose linked to long chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid is generally 16 or 18 carbon atoms and may contain one or more unsaturated bonds. Furthermore, the sophorose residue can be acetylated on the 6- and/or 6′-position(s). The fatty acid carboxyl group can be free (acidic or linear form (General Formula 2)) or internally esterified at the 4″-position (lactonic form (General Formula 1)). S. bombicola produces a specific enzyme, called S. bombicola lactone esterase, which catalyzes the esterification of linear SLP to produce lactonic SLP.

In preferred embodiments, the SLP according to the subject invention are represented by General Formula (1) and/or General Formula (2), and include 30 or more structural homologs:

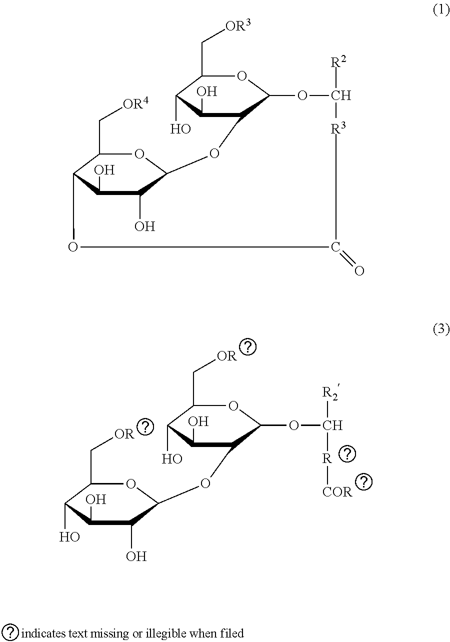

where R¹ and R^{1′} independently represent saturated hydrocarbon chains or single or multiple, in particular single, unsaturated hydrocarbon chains having 8 to 20, in particular 12 to 18 carbon atoms, more preferably 14 to 18 carbon atoms, which can be linear or branched and can comprise one or more hydroxy groups, R² and R^{2′} independently represent a hydrogen atom or a saturated alkyl functional group or a single or multiple, in particular single, unsaturated alkyl functional group having 1 to 9 carbon atoms, more preferably 1 to 4 carbon atoms, which can be linear or branched and can comprise one or more hydroxy groups, and R³, R^{3′}, R⁴ and R^{4′} independently represent a hydrogen atom or —COCH₃. R⁵ is typically, but is not limited to, —H or —OH.

The composition utilized according to the subject methods can comprises more than one form of SLP, including linear SLP and lactonic SLP. The SLP can be non-acetylated, mono-acetylated and/or di-acetylated SLP.

In certain specific embodiments, the composition comprises SLP according to General Formula (1) (linear SLP) wherein R¹ and/or R² are an acetyl group, and wherein R³ is derived from a stearic, oleic and/or linoleic fatty acid.

SLP are typically produced by yeasts, such as Starmerella spp. yeasts and/or Candida spp. yeasts, e.g., Starmerella (Candida) bombicola, Candida apicola, Candida batistae, Candida floricola, Candida riodocensis, Candida stellate and/or Candida kuoi. SLP have environmental compatibility, high biodegradability, low toxicity, high selectivity and specific activity in a broad range of temperature, pH and salinity conditions. Additionally, in some embodiments, SLP can be advantageous due to their small micelle size, which can help facilitate the movement of the micelle, and compounds enclosed therein, through nanoscale pores and spaces. In certain embodiments, the micelle size of a SLP is less than 100 nm, less than 50 nm, less than 20 nm, less than 15 nm, less than 10 nm, or less than 5 nm.

In certain embodiments, the glycolipid is a rhamnolipid. Rhamnolipids comprise a glycosyl head group (i.e., a rhamnose) moiety, and a 3-(hydroxyalkanoyloxy)alkanoic acid (HAA) fatty acid tail, such as, e.g., 3-hydroxydecanoic acid. Two main subtypes of rhamnolipids exist, mono- and di-rhamnolipids, which comprise one or two rhamnose moieties, respectively. The HAA moiety can vary in length and degree of branching, depending on, for example, the growth medium and the environmental conditions. The highest accumulation of rhamnolipids (RLP) has been shown by submerged cultivation of Pseudomonas spp., such as P. aeruginosa.

Rhamnolipids according to the subject invention can have the following structure, according to General Formula (3):

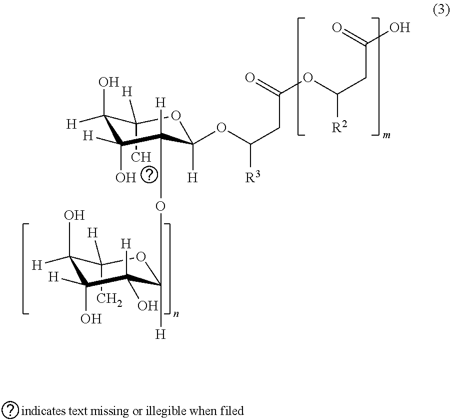

wherein m is 2, 1 or 0,
n is 1 or 0,
R¹ and R² are, independently of one another, the same or a different organic functional group having 2 to 24, preferably 5 to 13 carbon atoms, in particular a substituted or unsubstituted, branched or unbranched alkyl functional group, which can also be unsaturated,
wherein the alkyl functional group is a linear saturated alkyl functional group having 8 to 12 carbon atoms, or is a nonyl or a decyl functional group or a mixture thereof.

Salts of these compounds are also included according to the invention. In the present invention, the term “di-rhamnolipid” is understood to mean compounds of the above formula or the salts thereof in which n is 1. Accordingly, “mono-rhamnolipid” is understood in the present invention to mean compounds of the general formula or the salts thereof in which n is 0. In certain specific embodiments, the composition comprises a mixture of mono- and di-rhamnolipids. As used herein, “filtering” refers to the process by which a solid or semisolid particle is removed from a liquid, for example, by passing the liquid through a filter medium through which the solid or semisolid particle cannot pass.

As used herein, “beneficiation” refers to the process by which gangue materials are removed from the product of interest (e.g., element, compound, mineral).

As used herein, “ore” refers to a naturally occurring solid material from which a valuable substance, mineral and/or metal can be profitably extracted. Ores are often mined from ore deposits, which comprise ore minerals containing the valuable substance. “Gangue” minerals are minerals that occur in the deposit but do not contain the valuable substance. As used herein, “contaminant” can refer to gangue. Examples of ore deposits include hydrothermal deposits, magmatic deposits, laterite deposits, volcanogenic deposits, metamorphically reworked deposits, carbonatite-alkaline igneous related deposits, placer ore deposits, residual ore deposits, sedimentary deposits, sedimentary hydrothermal deposits and astrobleme-related deposits. Ores, as defined herein, however, can also include ore concentrates or tailings.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9% 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Precipitation Compositions

In certain embodiments, the subject invention provides compositions comprising components that are derived from microorganisms. In certain embodiments, the composition comprises a microbial biosurfactant. In certain embodiments, the composition comprises one or more biosurfactants, and, optionally, other compounds, such as, for example, water; alkali; sulfide; coagulant; chemical surfactants, including, for example, ionic surfactants or nonionic surfactants; flocculants; clarifying agents; filtration aids; or any combination thereof.

In certain embodiments, the alkali is, for example, sodium hydroxide (NaOH), hydrated lime (Ca(OH)₂), quick lime (CaO), limestone (CaCO₃), and magnesium hydroxide (Mg(OH)₂). In some embodiments, the alkali source can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total precipitation composition.

In certain embodiments, the sulfide reagent is, for example, iron sulfide (FeS), sodium hydrosulfide (NaHS), sodium sulfide (Na₂S), calcium sulfide (CaS), biogenic sulfide, optionally generated in situ by sulfate reaction, or any combination thereof. In some embodiments, the sulfide reagent can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total precipitation composition.

In certain embodiments, the coagulant is, for example, alum (KAl(SO₄)₂), iron hydroxide (Fe(OH)₃), ferric chloride (FeCl₃), or any combination thereof. In some embodiments, the coagulant can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total precipitation composition.

In certain embodiments, carbonates can also be used in precipitation, such as, for example, sodium carbonate (Na₂CO₃), calcium carbonate (CaCO₃), or carbon dioxide (CO₂), optionally under a pressure of 5.2 bar to 74 bar. In some embodiments, the carbonate can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total precipitation composition.

In certain embodiments, the clarifying agent is, for example, alum, aluminum chlorohydrate, aluminum sulfate, calcium oxide, calcium hydroxide, ferrous sulfate, ferric chloride, polyacrylamide, polyDADMAC (Polydiallyldimethylammonium chloride), sodium aluminate, sodium silicate, or any combination thereof. In certain embodiments, the carbonate can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total precipitation composition.

In certain embodiments, the chemical surfactant of the precipitation composition is a detergent, wetting agent, emulsifier, foaming agent, and/or dispersant. In certain embodiments, the ionic surfactants can be, for example, fatty alcohol sulfates (e.g., sodium dodecyl sulfate or ammonium dodecyl sulfate); fatty alcohol ether sulfates; alkyl sulfoacetates; fatty alcohol phosphoric acid esters; fatty alcohol ether phosphates; alcohol phosphoric acid esters, including, for example, triisobutyl phosphate, monoalkyl or dialkyl esters of sulfosuccinic acid (e.g., dioctyl sodium sulfosuccinate, alkyl sulfonates, alkylbenzenesulfonates, including, for example, dodecylbenzenesulfonic acid); and nonionic surfactants, including, for example, fatty alcohol ethoxylates (e.g., alkylphenol ethoxylates, polyoxyethylene fatty acid esters, polypropylene glycol ethoxylates, fatty acid mono- and diglycerides, fatty acid glycol partial esters, sorbitan fatty acid esters, or polyoxyethylene sorbitan fatty acid esters).

In certain embodiments, the filtration aids include, for example, conventional filtration aids, such as, for example, diatomaceous earth, charcoal, perlite, asbestos, cellulose, or fly ash. Filtration aids can be chemicals that assist in solid-liquid separation by modifying the surface properties of minerals, elements, or other substances to enhance water repellency. The filtration aids impart a hydrophobic character to particles so that interstitial water is reduced to a minimum. Flocculants or clarifying agents constitute types of filtration aids; by binding the particles of the precipitate together, they prevent the particles from binding to the filter medium and inhibiting the movement of the liquid through the filter medium.

In certain embodiments, the flocculant is, for example, ferric chloride, ferric sulfate, ferric chloride sulfate, ferrous sulfate, aluminum sulfate, aluminum chloride, sodium aluminate, aluminum chlorohydrate, polyaluminum chloride, polyacrylamide, sulfate, guar gum, alginates, strychnos potatorum seeds, starch, cellulose, tannin, chitosan, isinglass, gelatin, or any combination thereof.

In certain embodiments, the filtration aid is, for example, an adsorbent, such as a silica gel; natural glasses, including, for example, expanded perlite, pumice, expanded pumice, pumicite, expanded obsidian, and expanded volcanic ash; and biogenic silicas, such as, for example, diatomite, rice hull ash, and sponge spicules. In a further embodiment, the filtration aid may be, for example, buoyant glasses, buoyant polymers, and/or celluloses. In certain embodiments, the cellulose materials can have a porous filtration component that may result in buoyancy enhanced filtration.

In certain embodiments, the precipitation composition comprises a microbe-based product comprising a biosurfactant utilized in crude form. The crude form can comprise, in addition to the biosurfactant, fermentation broth in which a biosurfactant-producing microorganism was cultivated, residual microbial cell matter or live or inactive microbial cells, residual nutrients, and/or other microbial growth by-products. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

In some embodiments, the biosurfactant is utilized after being extracted from a fermentation broth and, optionally, purified.

The biosurfactant according to the subject invention can be a glycolipid (e.g., sophorolipids, rhamnolipids, cellobiose lipids, mannosylerythritol lipids and trehalose lipids), lipopeptide (e.g., surfactin, iturin, fengycin, arthrofactin and lichenysin), flavolipid, phospholipid (e.g., cardiolipins), fatty acid ester compound, fatty acid ether compound, and/or high molecular weight polymers such as lipoproteins, lipopolysaccharide-protein complexes, and polysaccharide-protein-fatty acid complexes.

In certain specific embodiments, the biosurfactant is a sophorolipid (SLP), including linear SLP, lactonic SLP, acetylated SLP, de-acetylated SLP, salt-form SLP derivatives, esterified SLP derivatives, amino acid-SLP conjugates, and other SLP derivatives or isomers that exist in nature and/or are produced synthetically. In preferred embodiments, the SLP is a linear SLP or a derivatized linear SLP. In certain embodiments, the subject compositions can comprise lactonic and linear SLP, with at least about 50%, about 55%, about 60%, about 65%, about 70%, about 75%, about 80%, about 85%, about 90%, or about 95% of the SLP comprising linear forms, and the remainder comprising lactonic forms.

In some embodiments, the biosurfactant can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total precipitation aid composition.

In another embodiment, a purified biosurfactant may be added in combination with an acceptable carrier, in that the biosurfactant may be presented at concentrations of 0.001 to 50% (v/v), preferably, 0.01 to 20% (v/v), more preferably, 0.02 to 5% (v/v).

In some embodiments, the biosurfactant can be included in the composition at, for example, 0.01 to 100,000 ppm, 0.05 to 10,000 ppm, 0.1 to 1,000 ppm, 0.5 to 750 ppm, 1.0 to 500 ppm, 2.0 to 250 ppm, or 3.0 to 100 ppm, with respect to the amount of liquid being treated.

In some embodiments, the conventional precipitation aid, flocculant, and/or clarifying agent can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total precipitation aid composition.

Conventional precipitation compositions comprise different types of precipitating agents that are commonly used to help a precipitate form. Examples of conventional precipitation aids include lime (CaO), limestone (CaCO₃), calcium hydroxide (Ca(OH)₂), sodium hydroxide (NaOH), or any combination thereof.

The subject composition can further comprise other additives such as, for example, carriers, other microbe-based compositions, additional biosurfactants, enzymes, catalysts, solvents, salts, buffers, chelating agents, acids, emulsifying agents, lubricants, solubility controlling agents, preservatives, stabilizers, ultra-violet light resistant agents, viscosity modifiers, preservatives, tracking agents, and other microbes and other ingredients specific for an intended use.

In certain embodiments, chelating agents can be, but are not limited to, ethylenediaminetetraacetic acid (EDTA), nitrilotriacetic acid (NTA), a phosphonate, succimer (DMSA), diethylenetriaminepentaacetate (DTPA), N-acetylcysteine, n-hydroxyethylethylenediaminetriacetic acid (HEDTA), organic acids with more than one coordination group (e.g., rubeanic acid), STPP (sodiumtripolyphosphate, Na5P3O10), trisodium phosphate (TSP), water, carbohydrates, organic acids with more than one coordination group (e.g., citric acid), lipids, steroids, amino acids or related compounds (e.g., glutathione), peptides, phosphates, nucleotides, tetrapyrrols, ferrioxamines, ionophores, orphenolics, sodium citrate, sodium gluconate, ethylenediamine disuccinic acid (EDDS), iminodisuccinic acid (IDS), L-glutamic acid diacetic Acid (GLDA), GLDA-Na₄, methyl glycindiacetic acid (MGDA), polyaspartic acid (PASA), hemoglobin, chlorophyll, lipophilic β-diketone, and (14,16)-hentriacontanedione, ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), pyridine-2,6-dicarboxylic acid (PDA), trimethyl glycine (TMG), Tiron, or any combination thereof.

### Methods of Precipitation

In certain embodiments, the subject invention provides a method for separating contaminants, minerals, elements, or other target compounds from a solution by precipitating the contaminants, target elements, target minerals, or other target compounds from the solution. This process can precipitate contaminants, target elements, target minerals, or other target compounds out of a liquid from various sources, including, for example, mining sites (e.g., slurry of ore or mining wastewater), quarrying sites, oil, and gas well recovery sites, mineral refineries, wastewater sites, agricultural sites, food and beverage production sites, and industrial sites.

In certain embodiments, the mining site can be a coal mine, iron ore mine (e.g., taconite), copper mine, copper-nickel mine, tin mine, nickel mine, gold mine, silver mine, molybdenum mine, aluminum mine (e.g., bauxite mine, kyanite mine), lead-zinc mine, tungsten mine, phosphate mine, potash mine, mica mine, bentonite mine, or zinc mine. The mine can be an underground mine, surface mine, placer mine or in situ mine. In certain embodiments, a variety of toxic compounds can be derived from mining activities. In certain embodiments, methods of removing said toxic compounds are provided according to the subject methods by contacting the precipitation compositions to various liquid streams, piping, pumps, liquid storage areas, or aquatic environments. The toxic compounds can include, for example, cyanide, sulfur-bearing minerals, soluble iron, and heavy metals, such as, for example, molybdenum, tungsten, chromium, manganese, nickel, arsenic, and vanadium. In certain embodiments, the quarrying site can extract chalk, clay, cinder, coal, sand, gravel, coquina, diabase, gabbro, granite, gritstone, gypsum, limestone, marble, ores, phosphate rock, quartz, sandstone, slate, travertine, or any combination thereof.

In certain embodiments, the methods of the subject invention can be used with traditional precipitation methods. Traditional precipitation methods use chemical precipitation to treat, for example, mining-influences water, such as, for example, acid mine drainage, neutral drainage, and pit lake water. Chemical precipitation can remove metals, fats, oils, and other organic substances from wastewater. In certain embodiments, precipitation can be used to remove contaminants from the water so that it can be recycled. In the subject methods, water softening can precede precipitation. Water softening can cause ions to settle out of a solution. Examples of water softeners include, for example, ammonia, borax, calcium hydroxide, sodium carbonate (soda ash), and trisodium phosphate. Advantageously, biosurfactants are environmentally-friendly and can be efficient at aiding in precipitate formation.

In certain embodiments, the subject precipitation method can be used to precipitate asphaltene out of a solution. Asphaltenes are naturally found dissolved in crude oil and can clog pipelines and interrupt the movement of petroleum; therefore, the subject methods can be used to inhibit clogged pipes used in the extraction and processing of crude oil.

In certain embodiments, the subject precipitation methods can be used to refine minerals by chemical precipitation including, for example, clays, carbonates, sulfates, halides, zeolites, or cherts.

Produced water is industrial wastewater from an oil or gas well, a hydraulic fracturing site, an offshore oil drilling rig, a chemical production plant, or a petrochemical production plant. Geological features that contain oil and gas can also contain water and naturally occurring toxic substances, including arsenic and radium. In certain embodiments, the subject compositions and methods can be used to separate the unwanted or toxic elements or compounds from the produced water so the water can be recycled.

In certain embodiments, the precipitation composition can be applied to the mining waste liquid directly. In some embodiments, mixing the subject invention with the mining waste can create a precipitate of the target elements, minerals, or other compounds to form and separate from the liquid. In certain embodiments, the subject invention can be applied directly to, for example, the clarifier or other vessel in which mining waste is treated.

In certain embodiments, the subject methods can be used in a precoating process, in which the precipitation composition may initially be applied to a surface before a liquid containing a contaminant, target element, target mineral, or other target compound is contacted to the surface. In certain embodiments, the subject methods can be used to precoat a clarifier or other equipment. In certain embodiments, the subject composition can be contacted to surfaces, such as, for example, metal, steel, iron, plastic, copper, wood, acrylic, cement, ceramic, brick, concrete, carbon fiber, fiberglass, marble, plaster, rubber, stone, rubber, neoprene, or any combination thereof. The composition can be applied to the surface by dipping, dunking, spraying, rubbing, wiping, or any combination thereof.

In certain embodiments, the precipitate formed can be salts, such as, for example, lithium, sodium, potassium, cesium, rubidium, nitrate, chloride, bromide, iodide, silver, sulfate, or hydroxide salts. In certain embodiments, the precipitate formed can comprise a metal ion, such as, for example, As³⁺, Bi³⁺, Cd²⁺, Cu²⁺, Hg²⁺, Sb³⁺, and Sn²⁺. In certain embodiments, the precipitate formed can be a lithium salt. Lithium salts comprise lithium carbonate, lithium acetate, lithium sulfate, lithium citrate, lithium orotate, and lithium gluconate. In some embodiments, lithium salts can be found in underground deposits of clay, mineral ore and brine, geothermal water, and seawater. In certain embodiments, the percent of recovery of the precipitate, based on the amount collected compared to the total amount of the reactants, is in the range of about 41.6% to about 45.5% at 50° C. and about 22.6% to about 25.6% at 25° C. In preferred embodiments, the precipitation of lithium salts occurs at about 50° C. to about 60° C. In some embodiments, the precipitate can be separated from the liquid before undergoing further processing and/or refinement. In other embodiments, the precipitate can be disposed.

In certain embodiments, the method of precipitation causes minerals, elements, or compounds that are either dissolved in a solution to settle out of solution as a solid precipitate, which can then be filtered, centrifuged, or otherwise separated from the liquid portion.

In certain embodiments, precipitation comprises direct precipitation, co-precipitation, or homogeneous precipitation. Direct precipitation describes a process in which only one type of cation is contained in the solution, such as, for example, the synthesis of yttria nano-powder from yttrium nitrate and ammonia water, in which yttria nano-powder disassociates to make yttrium, the only cation in solution. Co-precipitation is the simultaneous precipitation of more than one compound from a solution. Co-precipitation can occur by the formation of mixed crystals by absorption, occlusion, or mechanical entrapment. Homogeneous precipitation describes a reaction in which the precipitating agent is synthesized in the solution. For example, Pb²⁺ is precipitated homogeneously as PbCrO₄ by using bromate (BrO³⁻) to oxidize Cr³⁺ to CrO₂⁴.

In certain embodiments, precipitation reactions comprise precipitation in solution, precipitation in agar, and/or precipitation in agar with an electric field.

In certain embodiments, the subject invention provides a method of precipitation for mining wastewater. By removing the contaminants or other unwanted compounds from the water, the water can be recycled. In certain embodiments, the subject methods can be effective at removing heavy metals from wastewater.

In certain embodiments, the subject methods can be used to precipitate a target element, target minerals or other target compound. In certain embodiments, the target precipitate can comprise, for example, cobalt (e.g., erythrite, skytterudite, cobaltite, carrollite, linnaeite, and asbolite (asbolane)); copper (e.g., chalcopyrite, chalcocite, bomite, djurleite, malachite, azurite, chrysocolla, cuprite, tenorite, native copper and brochantite); gold (e.g., native gold, electrum, tellurides, calaverite, sylvanite and petzite); silver (e.g., sulfide argentite, sulfide acanthite, native silver, sulfosalts, pyrargyrite, proustite, cerargyrite, tetrahedrites); aluminum (e.g., bauxite, gibbsite, bohmeite, diaspore); antimony (e.g., stibnite); barium (e.g., barite, witherite); cesium (e.g., pollucite); chromium (e.g., chromite); iron (e.g., hematite, magnetite, pyrite, pyrrhotite, goethite, siderite); lead (e.g., galena, cerussite, anglesite); lithium (e.g., pegmatite, spodumene, lepidolite, petalite, amblygonite, lithium carbonate); magnesium (e.g., dolomite, magnesite, brucite, carnallite, olivine); manganese (e.g., hausmannite, pyrolusite, barunite, manganite, rhodochrosite); mercury (e.g., cinnabar); molybdenum (e.g., molybdenite); nickel (e.g., pentlandite, pyrrhotite, garierite); phosphorus (e.g., hydroxylapatite, fluorapatite, chlorapatite); platinum group (platinum, osmium, rhodium, ruthenium, palladium) (e.g., native elements or alloys of platinum group members, sperrylite); potassium (e.g., sylvite, langbeinite); rare earth elements (cerium, dysprosium, erbium, europium, gadolinium, holmium, lanthanium, lutetium, neodymium, praseodymium, samarium, scandium, terbium, thulium, ytterbium, yttrium) (e.g., bastnasite, monazite, loparite); sodium (e.g., halite, soda ash); strontium (e.g., celestite, strontianite); sulfur (e.g., native sulfur, pyrite); tin (e.g., cassiterite); titanium (e.g., scheelite, huebnerite-ferberite); uranium (e.g., uraninite, pitchblende, coffinite, carnotite, autunite); vanadium; zinc (e.g., sphalerite, zinc sulfide, smithsonite, hemimorphite); and zirconium (e.g., zircon).

Additional target elements, target minerals, or other target compounds include, for example, arsenic, bertrandite, bismuthinite, borax, colemanite, kernite, ulexite, sphalerite, halite, gallium, germanium, hafnium, indium, iodine, columbite, tantalite-columbite, rubidium, quartz, diamonds, garnets (almandine, pyrope and andradite), corundum, barite, calcite, clays, feldspars (e.g., orthoclase, microcline, albite); gemstones (e.g., diamonds, rubies, sapphires, emeralds, aquamarine, kunzite); gypsum; perlite; sodium carbonate; zeolites; chabazite; clinoptilolite; mordenite; wollastonite; vermiculite; talc; pyrophyllite; graphite; kyanite; andalusite; muscovite; phlogopite; menatite; magnetite; dolomite; ilmenite; wolframite; beryllium; tellurium; bismuth; technetium; potash; rock salt; sodium chloride; sodium sulfate; nahcolite; niobium; tantalum and any combination of such substances or compounds containing such substances.

### Production of Microbe-Based Products

In certain embodiments, the subject invention provides methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

The microorganisms can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In certain embodiments, the microbes are capable of producing amphiphilic molecules, enzymes, proteins and/or biopolymers. Microbial biosurfactants, in particular, are produced by a variety of microorganisms such as bacteria, fungi, and yeasts, including, for example, Agrobacterium spp. (e.g., A. radiobacter); Arthrobacter spp.; Aspergillus spp.; Aureobasidium spp. (e.g., A. pullulans); Azotobacter (e.g., A. vinelandii, A. chroococcum); Azospirillum spp. (e.g., A. brasiliensis); Bacillus spp. (e.g., B. subtilis, B. amyloliquefaciens, B. pumillus, B. cereus, B. licheniformis, B. firmus, B. laterosporus, B. megaterium); Blakeslea; Candida spp. (e.g., C. albicans, C. rugosa, C. tropicalis, C. lipolytica, C. torulopsis); Clostridium (e.g., C. butyricum, C. tyrobutyricum, C. acetobutyricum, and C. beijerinckii); Campylobacter spp.; Cornybacterium spp.; Cryptococcus spp.; Debaryomyces spp. (e.g., D. hansenii); Entomophthora spp.; Flavobacterium spp.; Gordonia spp.; Hansenula spp.; Hanseniaspora spp. (e.g., H. uvarum); Issatchenkia spp; Kluyveromyces spp.; Meyerozyma spp. (e.g., M. guilliermondii); Mortierella spp.; Mycorrhiza spp.; Mycobacterium spp.; Nocardia spp.; Pichia spp. (e.g., P. anomala, P. guilliermondii, P. occidentalis, P. kudriavzevii); Phycomyces spp.; Phythium spp.; Pseudomonas spp. (e.g., P. aeruginosa, P. chlororaphis, P. putida, P. florescens, P. fragi, P. syringae); Pseudozyma spp. (e.g., P. aphidis); Ralslonia spp. (e.g., R. eulropha); Rhodococcus spp. (e.g., R. erythropolis); Rhodospirillum spp. (e.g., R. rubrum); Rhizobium spp.; Rhizopus spp.; Saccharomyces spp. (e.g., S. cerevisiae, S. boulardii sequela, S. torula); Sphingomonas spp. (e.g., S. paucimobilis); Starmerella spp. (e.g., S. bombicola); Thraustochytrium spp.; Torulopsis spp.; Ustilago spp. (e.g., U. maydis); Wickerhamomyces spp. (e.g., W. anomalus); Williopsis spp.; and/or Zygosaccharomyces spp. (e.g., Z. bailii).

In preferred embodiments, microorganism is a Starmerella spp. yeast and/or Candida spp. yeast, e.g., Starmerella (Candida) bombicola, Candida apicola, Candida batistae, Candida floricola, Candida riodocensis, Candida stellate and/or Candida kuoi. In a specific embodiment, the microorganism is Starmerella bombicola, e.g., strain ATCC 22214.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites (e.g. small molecules and excreted proteins), residual nutrients and/or intracellular components (e.g. enzymes and other proteins).

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of organisms in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In some embodiments, the method for cultivation may further comprise adding additional acids and/or antimicrobials in the medium before, and/or during the cultivation process. Antimicrobial agents or antibiotics are used for protecting the culture against contamination.

Additionally, antifoaming agents may also be added to prevent the formation and/or accumulation of foam during submerged cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The microbial growth by-product produced by microorganisms of interest may be retained in the microorganisms or secreted into the growth medium. The medium may contain compounds that stabilize the activity of microbial growth by-product.

The biomass content of the fermentation medium may be, for example, from 5 g/l to 180 g/l or more, or from 10 g/l to 150 g/l.

The cell concentration may be, for example, at least 1×10⁶ to 1×10¹², 1×10⁷ to 1×10¹¹, 1×10⁸ to 1×10¹⁰, or 1×10⁹ CFU/ml.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

In certain embodiments, the subject invention provides a “microbe-based composition,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. The microbes may be present in or removed from the composition. The microbes can be present, with broth in which they were grown, in the microbe-based composition. The cells may be present at, for example, a concentration of at least 1×10³, 1×10⁴, 1×10⁵, 1×10⁶, 1×10⁷, 1×10⁸, 1×10⁹, 1×10¹⁰, 1×10¹¹, 1×10¹², 1×10¹³ or more CFU per milliliter of the composition.

The subject invention further provides “microbe-based products,” which are products that are to be applied in practice to achieve a desired result. The microbe-based product can be simply a microbe-based composition harvested from the microbe cultivation process. Alternatively, the microbe-based product may comprise further ingredients that have been added. These additional ingredients can include, for example, stabilizers, acids, buffers, catrers, such as water, salt solutions, or any other appropriate carrier, added nutrients to support further microbial growth, non-nutrient growth enhancers, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

One microbe-based product of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based products may also contain a combination of any of these forms of a microorganism.

In one embodiment, different strains of microbe are grown separately and then mixed together to produce the microbe-based product. The microbes can, optionally, be blended with the medium in which they are grown and dried prior to mixing.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, ultra-violet light resistant agents, other microbes and other suitable additives that are customarily used for such preparations.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 200 C, 15° C., 10° C., or 5° C. On the other hand, a biosurfactant composition can typically be stored at ambient temperatures.

1. A method of precipitation, the method comprising:
a) contacting a precipitation composition comprising a biosurfactant with an aqueous solution containing at least one charged ion; and
b) separating the precipitated solid from the liquid.

2. The method of claim 1, wherein step b) further comprises filtering, centrifugation, settling, or a combination thereof.

3. (canceled)

4. The method of claim 1, wherein the at least one charged ion is a nitrate, chloride, bromide, iodide, sulfate, lithium or hydroxide.

5. (canceled)

6. The method of claim 4, wherein the lithium salt is lithium carbonate, lithium acetate, lithium sulfate, lithium citrate, lithium orotate, lithium gluconate, and any combination thereof.

7. (canceled)

8. The method of claim 1, wherein the aqueous solution comprises a wastewater or slurry of ore, wherein the wastewater comprises municipal wastewater, stormwater, mining wastewater, quarrying wastewater, groundwater, and/or industrial wastewater.

9. (canceled)

10. The method of claim 8, wherein the wastewater or slurry of ore is from a coal mine, iron ore mine, copper mine, copper-nickel mine, tin mine, nickel mine, gold mine, silver mine, molybdenum mine, aluminum mine, lead-zinc mine, tungsten mine, phosphate mine, potash mine, mica mine, bentonite mine, or zinc mine.

11. (canceled)

12. The method of claim 8, wherein the industrial wastewater is produced water from an oil or gas well, a hydraulic fracturing site, an offshore oil drilling rig or water from a chemical production plant or petrochemical production plant.

13-15. (canceled)

16. The method of claim 1, wherein the biosurfactant is a glycolipid.

17. (canceled)

18. The method of claim 61, wherein the precipitation composition comprises a glycolipid selected from sophorolipids, mannosylerythritol lipids, trehalose lipids, and rhamnolipids.

19. (canceled)

20. A precipitation composition comprising a glycolipid and/or an inactive yeast culture comprising a glycolipid and one or more traditional precipitation components; wherein the yeast culture is a Starmerella sp. and/or a Candida sp. yeast; wherein the glycolipid is a sophorolipid, mannosylerythritol lipid, trehalose lipid, rhamnolipid, or any combination thereof; and wherein the one or more traditional precipitation components are selected from one or a combination of the following:
a) chemical surfactant;
b) flocculant;
c) filtration aid;
d) clarifying agent;
e) alkali;
f) sulfide;
g) coagulant; and
h) carbonate.

21-24. (canceled)

25. The composition of claim 20, comprising a chemical surfactant selected from ammonium lauryl sulfate, sodium laureth sulfate, sodium lauryl sarcosinate, sodium myreth sulfate, sodium pareth sulfate, sodium stearate, and sodium lauryl sulfate.

26. The composition of claim 20, comprising a flocculant selected from ferric chloride, ferric sulfate, ferrous sulfate, ferric chloride sulfate, aluminum sulfate, ferric sulfate, aluminum sulfate, aluminum chloride, polyacrylamide, sulfate, sodium aluminate, chloride, aluminum chlorohydrate, chitosan, isinglass, Moringa oleifera seeds, gelatin, strychnos potatorum seeds, guar gum, and alginates.

27. The composition of claim 20, comprising a filtration aid selected from diatomaceous earth, charcoal, perlite, asbestos, cellulose, fly ash, an absorbent, expanded perlite, pumice, expanded pumice, pumicite, expanded obsidian, expanded volcanic ash, biogenic silica, diatomite, rice hull ash, sponge spicule, buoyant polymer, and cellulose material.

28. The composition of claim 20, comprising a clarifying agent selected from alum, aluminum chlorohydrate, aluminum sulfate, calcium oxide, calcium hydroxide, ferrous sulfate, ferric chloride, polyacrylamide, polyDADMAC (Polydiallyldimethylammonium chloride), sodium aluminate, and sodium silicate.

29. The composition of claim 20, comprising a alkali selected from sodium hydroxide (NaOH), hydrated lime (Ca(OH)₂), quick lime (CaO), limestone (CaCO₃), and magnesium hydroxide (Mg(OH)₂).

30. The composition of claim 20, comprising a sulfide selected from iron sulfide (FeS), sodium hydrosulfide (NaHS), sodium sulfide (Na₂S), calcium sulfide (CaS), and biogenic sulfide.

31. The composition of claim 30, wherein biogenic sulfide is generated in situ by a sulfate reaction.

32. The composition of claim 20, comprising a coagulant selected from alum (KAl(SO₄)₂), iron hydroxide (Fe(OH)₃), and ferric chloride (FeCl₃).

33. The composition of claim 20, comprising a carbonate selected from sodium carbonate (Na₂CO₃), calcium carbonate (CaCO₃), and carbon dioxide (CO₂).

34. The composition of claim 33, wherein carbon dioxide is under a pressure of 5.2 bar to 74 bar.
